(19) United States
(12) Patent Application Publication
(10) Pub. No.: US 2008/0234291 A1
(43) Pub. Date: Sep. 25, 2008

(54) Mitratapide Oral Solution

(76) Inventors: Marc Karel Jozef Francois, Kapellen (BE); Roger Carolus Augusta Embrechts, Oud-Turnhout (BE); Willy Maria Albert Carlo Dries, Merksplas (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/585,754

(22) PCT Filed: Jan. 18, 2005
(86) PCT No.: PCT/EP2005/050181

§ 371 Date: Jul. 12, 2006

(30) Foreign Application Priority Data
Jan. 21, 2004 (EP) 04100177.7

(51) Int. Cl. A61K 31/496; A61P 3/00

(52) U.S. Cl. 514/254.07

(57) ABSTRACT

The present invention concerns an oral solution comprising the MTP inhibitor mitratapide or an pharmaceutically acceptable acid addition salt thereof, a process for preparing such solutions, and their use in the treatment of MTP-related disorders such as hyperlipidemia, obesity, or type II diabetes.

The present invention concerns an oral solution comprising the MTP inhibitor mitratapide or a pharmaceutically acceptable acid addition salt thereof, a process for preparing such solutions, and their use in the treatment of MTP-related disorders such as hyperlipidemia, obesity, or type II diabetes.

Mitratapide is the International Non Proprietary (INN) name for the compound (−)-[2S-[2α,4α(S*)]]-4-[4-[4-[4-[[2-(4-chlorophenyl)-2-[[(4-methyl-4H-1,2,4-triazol-3-yl)thio]methy]-1,3-dioxolan-4-yl]methoxy]pheny]-1-piperazinyl]phenyl]-2,4-dihydro-2-(1-methylpropyl)-3H-1,2,4-triazol-3-one having the following structure.

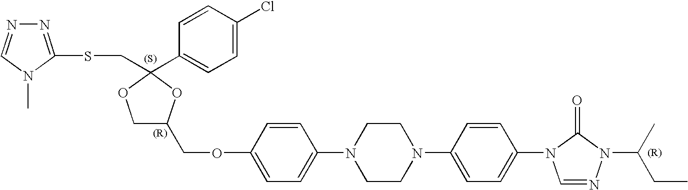

Mitratapide has been described in WO-96/13499 as compound 40 having apolipo-protein B (apoB) secretion and microsomal triglyceride transfer protein (MTP) inhibiting properties and therefore useful as a lipid lowering agent.

WO-99/22738 discloses melt-extruded particles comprising mitratapide as a lipid lowering agent and water-soluble polymers. WO-99/55313 discloses sugar sphere pellets coated with a film of a water-soluble polymer and mitratapide as a lipid lowering agent, and a seal coating layer.

The oral solutions of the present invention are particularly useful for the treatment of obese warm-blooded animals, in particular companion animals, especially dogs and cats. Companion animals with an excessive accumulation of body fat to the point of being 20% more over ideal body weight are considered obese. Obesity is known to cause liver disease, hypertension, constipation, heat intolerance, and increased risk under anesthesia. Obese companion animals may have trouble breathing and may suffer from serious discomfort and body dysfunction and do not have life expectancies as long as usual. Although obesity in companion animals is usually caused by too little exercise and too many calories, a number of pets become obese due to genetic predisposition or hormonal disorders.

The use of a solution for oral administration to animals is preferred as it is convenient and the dosage can be accurately controlled. In combination with an appropriate metering system, e.g. calibrated syringes or pipettes, an oral solution provides high flexibility in controlling the dosage. This facilitates administration to animal species of different sizes or to different animal species or breeds, with varying dosage requirements. Additionally, an oral solution allows the use of flavouring and/or palatability agents that can promote animal acceptance and compliance, which can be particularly advantageous when dosing chronically to animals.

Solutions comprising mitratapide suitable for oral administration have been described in Atherosclerosis vol. 144 (Supplement 1), page 39 (1999), WO-99122738 page 7, lines 9-11, and WO-99155313, page 7, lines 10-13, as an aqueous solution further comprising cyclodextrin derivatives as a solubilizing agent.

Since mitratapide has a solubility of less than 0.5 μg/ml in water, which can be increased to 0.4 mg/ml at a pH of 1.2, the presence of a solubilizing agent is necessary for the preparation of aqueous solutions. Hydroxypropyl-β-cyclodextrin (HP-β-CD) is a suitable solubilizing agent with which mitratapide forms higher order water soluble complexes. In order to obtain an aqueous solution comprising mitratapide with a concentration of 5 mg/ml it is necessary to use HP-β-CD in an amount of 250 mg/ml and adjusting the pH to 4. Furthermore it is necessary to add an antimicrobial preservative to these aqueous HP-β-CD solutions to protect them against microbial spoilage during manufacture or use. The addition of benzoic acid, a well known antimicrobial preservative for acidic aqueous solutions having a pH ≦4.5, however caused a precipitation of mitratapide probably due to a competition between mitratapide and benzoic acid for inclusion into the HP-β-CD cavity. To compensate, the HP-β-CD concentration had to be increased from 250 mg/ml to 400 mg/ml to solubilize mitratapide at a concentration of 5 mg/ml. Furthermore the antimicrobial activity of benzoic acid was decreased by the inclusion of some of the benzoic acid in HP-β-CD, resulting in a failure to meet the requirements of the European Pharmacopoeia for the antimicrobial efficacy test, even at a total benzoic acid concentration of 5 mg/ml.

Because aqueous mitratapide solutions require a high amount of the very expensive HP-β-CD and fail to meet the requirements of the European Pharmacopoeia for the antimicrobial efficacy test (AET) despite the use of very high benzoic acid concentrations, there is a need to develop mitratapide solutions suitable for oral administration to animals that are stable, easy to use and meet the requirements of the AET.

It has now been found that solutions comprising mitratapide or a pharmaceutically acceptable salt thereof, a pharmaceutically acceptable solvent wherein mitratapide has a solubility of 5 mg/ml or higher at a temperature of 22° C., a taste modifying agent and an antioxidant, fulfil these requirements.

The pharmaceutically acceptable salts of mitratapide are acid addition salt forms of mitratapide obtained by treating mitratapide in its base form with an appropriate inorganic or organic acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids.

The pharmaceutically acceptable solvent wherein mitratapide has a solubility of 5 mg/ml or higher is preferably selected from the group consisting of dimethyl isosorbide, diethylene glycol monoethyl ether, caprylocaproyl macrogol-8 glyceride, propylene glycol monolaurate, polyethyleneglycol 200, polyethyleneglycol 300 and polyethyleneglycol 400, or mixtures thereof, or mixtures of polyethylene glycols (PEGs) having an average molecular weight higher than 400 with PEGs having an average molecular weight lower than 400 so that the mixture thereof is liquid at room temperature. PEGs with an average molecular weight higher than 400, e.g. PEG 600, PEG 900, PEG 1000, PEG 1500 and the like, are solid at room temperature. By mixing these PEGs with a PEG such as e.g. PEG 100, PEG 200 or PEG 300, a mixture can be obtained that is fluid at room temperature.

The solubility of mitratapide in different pharmaceutical solvents was measured at room temperature of about 22° C. and listed in Table 1.

TABLE 1
solubility of mitratapide in mg/ml

| | solubility |
|---|---|
| Solvent | (mg/ml) |
| glycerol | <0.02^{a} |
| sesame oil | <0.05 |
| caprylic/capric acid triglyceride (Miglyol | <5 |
| 812 ®) | |
| caprylic/capric/succinic acid triglyceride | <5 |
| (Miglyol 829 ®) | |
| caprylidcapridlinoleic acid triglyceride | <5 |
| (Miglyol 818 ®) | |
| apricot Kernel oil PEG-6 complex (Labrafil | <5 |
| 1944CS ®) | |
| corn oil PEG-6 complex (Labrafil 2125CS ®) | <5 |
| caprylic/capric diester of propylene glycol | <5 |
| propyleneglycol | 2.2^{a} |
| dimethyl isosorbide (2,5-di-O-methyl- | >5 |
| 1,4:3,6-dianhydro D-glucitol) | |
| diethylene glycol monoethyl ether | >5 |
| (Transcutol ®) | |
| caprylocaproyl-8 glyceride (Labrasol ®) | >5 |
| Dropylene glycol monolaurate (Lauroglycol ®) | >5 |
| polyethyleneglycol 400 (PEG 400) | 24.8^{a} |

^{a}per g solution

Labrasol®, Transcutol® and Lauroglycol® are commercially available from Gattefossé S. A., 92632 Gennevilliers Cedex, France. Miglyol® 812, 829, and 818 are available from Sasol Germany GmbH.

As demonstrated in Table 1 mitratapide can be solubilized in different pharmaceutically acceptable solvents at a concentration of 5 mg/ml or higher. Of these solvents, i.e. dimethyl isosorbide, diethylene glycol monoethyl ether, caprylocaproyl macrogol-8 glyceride, propylene glycol monolaurate and PEG 400, the latter is the most widely used in pharmaceutical drug products.

A 5 mg/ml mitratapide solution in PEG 400 has a moderately bitter taste and causes a burning sensation to the mouth. This bitter taste and burning mouth sensation were strongly reduced by the addition of a taste modifying agents.

Taste modifying agents suitable for use in the oral solutions of the present invention include: intense sweeteners, bulk sweeteners, flavouring agents, and taste masking agents. Examples of intense sweeteners are saccharin, aspartame, acesulfame, cyclamate, alitame, a dihydrochalcone sweetener, monellin, neohesperidin, neotame, stevioside or sucralose (4,1′,6′-trichloro-4,1′,6′-trideoxygalactosucrose), and the pharmaceutically acceptable salts thereof such as sodium or calcium saccharin, acesulfame potassium or sodium cyclamate. A preferred intense sweetener is sucralose. Examples of bulk sweeteners are sorbitol, mannitol, fructose, sucrose, maltose, isomalt, glucose, hydrogenated glucose syrup, xylitol, caramel or honey. Examples of flavouring agents are cherry, raspberry, black currant, strawberry flavour, caramel chocolate flavour, mint cool flavour, fantasy flavour, meat flavours and the like.

The taste modifying agent is preferably an intense sweetener conveniently employed in low concentrations ranging from 0.1 to 10 mg/ml depending on the sweetening properties of the intense sweetener. For example in the case of sucralose, which has 600 times the sweetness of sucrose, the concentration may range from 0.5 to 5 mg/ml, and preferably is 2 mg/ml.

The antimicrobial effectiveness of a 5 mg/ml mitratapide solution in PEG 400 further comprising 2 mg/ml sucralose was measured according to European Pharmacopoeia guidelines and compared with the antimicrobial effectiveness of an identical solution which further comprised one of the antimicrobial agents selected from methyl paraben, propyl paraben, butyl paraben, and benzoic acid. A statistical analysis on the antimicrobial efficacy test results demonstrated that neither the three paraben esters nor the benzoic acid had any effect. The vehicle itself, i.e. PEG 400, reduced microbial growth consistently which resulted in an oral solution that was safe to use with regard to resistance towards microbial contamination by micro-organisms.

Polyethylene glycols are known to exhibit some oxidizing activity due to the presence of small amounts of peroxide impurities. Therefore a one-month stability test was performed on a 5 mg/ml mitratapide solution in PEG 400 further comprising 2 mg/ml sucralose in the presence or absence of 0.5 mg/ml of the antioxidant BHT (butylated hydroxytoluene). The two solutions were stored at a temperature of 5° C., 25° C. and 40° C. The concentration of mitratapide and the total amount of impurities were measured at the start and after one month. The test results are summarized in Table 2.

TABLE 2
one month stability of a 5 mg/ml mitratapide solution
in PEG 400, further comprising 2 mg/ml sucralose,
in absence or presence of 0.5 mg/ml BHT

| | | | 5° C. | 25° C. | 40° C. |
|---|---|---|---|---|---|
| | Compound | At start | After one month | | |
| no BHT | total | 0.42% | 0.54% | 1.08% | 3.27% |
| | amount of | | | | |
| | impurities | | | | |
| 0.5 mg/ml | total | 0.42% | 0.40% | 0.54% | 1.08% |
| BHT | amount of | | | | |
| | impurities | | | | |

The stability of mitratapide in PEG 400 solutions was clearly improved by the addition of 0.5 mg/ml BHT. Further test were performed to evaluate the influence on the stability of mitratapide PEG 400 solutions with other antioxidants such as BHA (butylated hydroxyanisole), propyl gallate, DL-a-tocopherol (vitamin E), citric acid, and mixtures thereof. On the basis of a statistical analysis BHA was considered as the preferred antioxidant.

The antioxidant such as BHA (butylated hydroxyanisole), BHT (butylated hydroxytoluene), propyl gallate, DL-α-tocopherol (vitamin E), citric acid, or mixtures thereof, is present in amount ranging from 0.1 mg/ml to 10 mg/ml, preferably from 1 mg/ml to 5 mg/ml, more preferably 2 mg/ml.

A preferred oral solution of the present invention contains:

| | | |
|---|---|---|
| mitratapide | 5 | mg/ml |
| butylated hydroxyanisole (BHA) | 2 | mg/ml |
| sucralose | 2 | mg/ml |
| PEG 400 | 1 | ml (q.s.) |

In view of the apoB inhibiting activity and concomitant MTP inhibiting activity of mitratapide, the oral solutions of the present invention are suitable for the treatment and prevention of hyperlipidemia, hypercholesterolemia and hypertriglyceridemia and diseases associated therewith, e.g. cardiovascular diseases including cardiac ischemia, as well as obesity, pancreatitits and diabetes in warm-blooded animals, in particular companion animals, especially dogs and cats.

Accordingly the present invention also provides oral solutions comprising mitratapide for the manufacture of a medicament for treating or preventing hyperlipidemia, hypercholesterolemia and hypertriglyceridemia and diseases associated therewith, e.g. cardiovascular diseases including cardiac ischemia, as well as obesity, pancreatitits and diabetes in warm-blooded animals, in particular companion animals, especially dogs and cats.

The present invention further provides a method of treating a condition selected from hyperlipidemia, hypercholesterolemia, hypertriglyceridemia, obesity, pancreatitis, and diabetes which comprises administering to an animal in need of such treatment an oral solution of the present invention comprising a therapeutically effective amount of mitratapide. The method of treating diabetes also includes the treatment of insulin dependent diabetes mellitus (Type I) and non-insulin dependent diabetes mellitus (Type II).

The term “therapeutically effective amount of mitratapide” as used herein, means that amount of mitratapide that elicits the biological or medicinal response in the animal that is being sought by the veterinarian, which includes alleviation of the symptoms of the condition being treated. The therapeutically effective amount can be determined using routine optimization techniques and is dependent upon the particular condition to be treated, the condition of the animal, the route of administration, the formulation, and the judgment of the practitioner and other factors evident to those skilled in the art. A therapeutically effective amount may be achieved by multiple dosing.

The dosage in vivo may range between 0.1 mg/kg and 10 mg/kg, particular between 0.3 mg/kg and 3 mg/kg, more particular 0.63 mg/kg.

The oral solutions of the present invention can be administered directly in the oral cavity or more preferably mixed with the food. Dosing of the oral solution can be done using an appropriate metering system such as e.g. a calibrated syringe, pipette, or a pre-filled dispenser that can deliver calibrated amounts of fluid.

The oral solutions of the present invention can be prepared by dissolving mitratapide, the intense sweetener and the antioxidant in the pharmaceutically acceptable solvent wherein mitratapide has a solubility of 5 mg/ml or higher at a temperature of 22° C. and stirring until a homogeneous solution is obtained. Optionally colloid-milling is used to aid the dissolution of mitratapide. A pharmaceutical dosage form is obtained by filtering the previous solution and filling it into suitable containers. e.g. in 100 ml glass bottles.

1. An oral solution comprising mitratapide or a pharmaceutically acceptable salt thereof, a pharmaceutically acceptable solvent wherein mitratapide has a solubility of 5 mg/ml or higher at a temperature of 22° C., a taste modifying agent and an antioxidant.

2. An oral solution as claimed in claim 1 wherein the pharmaceutically acceptable solvent is selected from the group consisting of dimethyl isosorbide, diethylene glycol monoethyl ether, caprylocaproyl macrogol-8 glyceride, propylene glycol monolaurate, polyethyleneglycol 200, polyethyleneglycol 300 and polyethyleneglycol 400, and mixtures thereof, or mixtures of polyethylene glycols (PEGs) having an average molecular weight higher than 400 with PEGs having an average molecular weight lower than 400 so that the mixture thereof is liquid at room temperature.

3. An oral solution as claimed in claim 2 wherein the pharmaceutically acceptable solvent is polyethyleneglycol 400.

4. An oral solution as claimed in claim 1 wherein the taste modifying agent is an intense sweetener, a bulk sweetener, a flavouring agent, or a taste masking agent.

5. An oral solution as claimed in claim 4 wherein the taste modifying agent is an intense sweetener selected from the group consisting of saccharin, aspartame, acesulfame, cyclamate, alitame, a dihydrochalcone sweetener, monellin, neohesperidin, neotame, stevioside or sucralose (4,1′,6′-trichloro-4,1′,6′-trideoxygalactosucrose), and the pharmaceutically acceptable salts thereof.

6. An oral solution as claimed in claim 5 wherein the intense sweetener is present in an amount ranging from 0.1 to 10 mg/ml.

7. A oral solution as claimed in claim 6 wherein the intense sweetener is sucralose.

8. An oral solution as claimed in claim 1 wherein the antioxidant is selected from the group consisting of BHA, BHT, propyl gallate, DL-a-tocopherol, and citric acid, and mixtures thereof.

9. An oral solution as claimed in claim 8 wherein the antioxidant is present in an amount ranging from 0.1 to 10 mg/ml.

10. An oral solution as claimed in claim 9 wherein the antioxidant is BHA.

11. An oral solution as claimed in claim 10 comprising 5 mg/ml mitratapide, sucralose in an amount ranging from 0.5 to 5 mg/ml, and BHA in an amount ranging from 1 mg/ml to 5 mg/ml, dissolved in PEG 400.

12. An oral solution as claimed in claim 11 comprising 5 mg/ml mitratapide, sucralose in an amount of 2 mg/ml, and BHA in an amount of 2 mg/ml, dissolved in PEG 400.

13. A process of preparing an oral solution as claimed in claim 1, characterized in that said process comprises the steps of dissolving mitratapide, the taste modifying agent and the antioxidant in the pharmaceutically acceptable solvent wherein mitratapide has a solubility of 5 mg/ml or higher at a temperature of 22° C., and stirring until a homogeneous solution is obtained.
